(19) United States
(12) Patent Application Publication
(10) Pub. No.: US 2009/0171419 A1
(43) Pub. Date: Jul. 2, 2009

(54) CAPACITOR REFORMATION METHOD AND DEVICE CONFIGURED TO PERFORM THE CAPACITOR REFORMATION METHOD

(76) Inventors: Anthony W. Rorvick, Champlin, MN (US); Paul M. Skarstad, Plymouth, MN (US)

Correspondence Address: MEDTRONIC, INC., 710 MEDTRONIC PARKWAY NE, MINNEAPOLIS, MN, 55432-9924, US

(21) Appl. No.: 12/367,118

(22) Filed: Feb. 6, 2009

Related U.S. Application Data
(63) Continuation of application No. 11/209,485, filed on Aug. 23, 2005, now abandoned.

(51) Int. Cl. A61N 1/08

(52) U.S. Cl. 607/59

(57) ABSTRACT

A method for reforming one or more capacitors in an implantable medical device includes the steps of determining a capacitor reformation time period using empirical capacitor charging data, configuring the programming module with capacitor reformation instructions that include the capacitor reformation voltage and time period, and in response to the capacitor reformation instructions, charging the capacitors for the capacitor reformation time period.

### CROSS REFERENCE TO RELATED APPLICATION

This application is a continuation of U.S. application Ser. No. 11/209,485 filed on Aug. 23, 2005, incorporated by reference in this application.

### FIELD OF THE INVENTION

The present invention generally relates to capacitors, and to devices that utilize capacitors to store energy. More particularly, the present invention relates to implantable medical devices that provide an electrical therapy to a patient using the energy stored in the capacitors.

### BACKGROUND OF THE INVENTION

Implantable cardioverter-defibrillators are used to treat patients suffering from heart rhythm irregularities. In operation, a defibrillator device monitors the electrical activity of a patient's heart, detects any arrhythmias, and delivers an appropriate corrective electrical therapy. Specifically one or more bursts of electric current are delivered in response to the arrhythmias.

A typical cardiac defibrillator or cardioverter includes a set of electrical leads, which extend from a sealed housing into a patient's heart after implantation. Within the housing area battery for supplying power, a capacitor for delivering bursts of electric current through the leads to the heart, and monitoring circuitry for monitoring the heart and determining an appropriate electrical therapy. The monitoring circuitry generally includes a microprocessor and a memory that stores instructions not only dictating how the microprocessor answers therapy questions, but also controlling certain device maintenance functions, such as maintenance of the capacitors in the apparatus.

As much as 30 to 35 joules may be delivered from capacitors that are configured to provide energy to a patient in a fraction of a second. However, typical cardioverter-defibrillators require capacitor maintenance in order for the capacitors to operate with full efficiency. One maintenance issue with electrolytic capacitors concerns charging efficiency degradation after long periods of inactivity. Reduced efficiency, which commonly stems from degradation of the dielectric oxide, requires a power source to progressively spend additional time and energy to charge the capacitors. The process of restoring full efficiency to an electrolytic capacitor is called “reformation.” Capacitors have previously been reformed by various methods that include the steps of charging the capacitors to a specified high voltage, holding the high voltage for a period of time, and then either discharging the capacitors through a non-therapeutic load or allowing them to “bleed down” by way of an open circuit. However, such capacitor reforming methods require special circuitry and decision-making logic.

Accordingly, it is desirable to provide methods of reforming capacitors in an implantable medical device to thereby overcome problems associated with degraded charging efficiency. In addition, it is desirable to provide methods of reforming capacitors that are efficient in terms of power consumption, and that do not require an invasive procedure with respect to the patient's body. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A method is provided for reforming one or more capacitors in an implantable medical device having a control system that includes a programming module, wherein each capacitor in the implantable medical device has a maximum rated voltage. First, using empirical capacitor charging data, a capacitor reformation time period is determined. Then, the programming module is configured with capacitor reformation instructions that include the capacitor reformation time period. In response to the capacitor reformation instructions, the capacitors are charged for the capacitor reformation time period, to a voltage that is at least 20% and less than 90% of the maximum-charging voltage.

An implantable medical device is also provided. The implantable medical device comprises a control system having a processor and a programming module configured with capacitor reformation instructions that include a fixed capacitor reformation time period during which the capacitors are to be charged to a reformation voltage that is at least 20% and less than 90% of the maximum-charging voltage. The device further comprises a lead system comprising at least one lead, and a therapy system, comprising a plurality of capacitors having a maximum-charging voltage, and coupled to receive the capacitor reformation instructions and, responsive thereto, to charge the plurality of capacitors for the fixed capacitor reformation time period, to the reformation voltage.

### BRIEF DESCRIPTION OF THE DRAWINGS

The present invention will hereinafter be described in conjunction with the following drawing figures, wherein like numerals denote like elements, and

FIG. 1 is an illustration of an ICD and a lead system that extends to a patient's heart;

FIG. 2 is a block diagram illustrating an ICD system in which the present invention can be incorporated;

FIG. 3 is a cross-sectional view of an exemplary capacitor for which the reformation method and system of the present invention can be incorporated;

FIG. 4 is a block diagram representing an implantable cardiac device according to an exemplary embodiment of the invention; and

FIG. 5 is a block diagram that outlines a method of scheduling and performing capacitor reformation maintenance for an ICD according to an exemplary embodiment of the invention.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The following description is set forth with reference to an implantable cardiac device (ICD), although the principles of the invention may be implemented in any pertinent implantable medical device employing a transformer to provide therapy and/or monitoring functions.

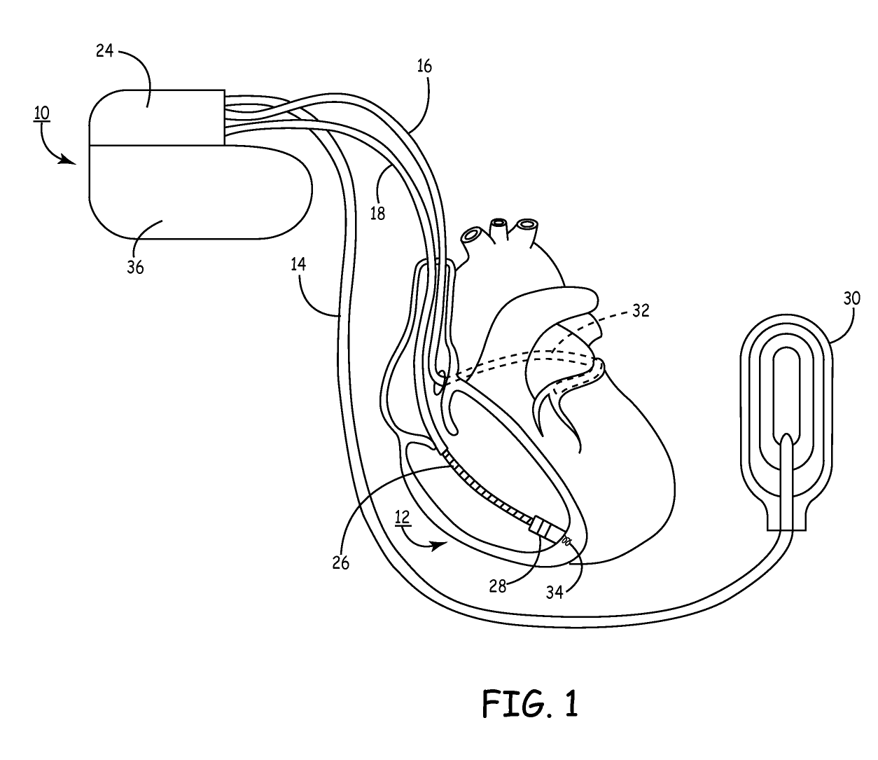

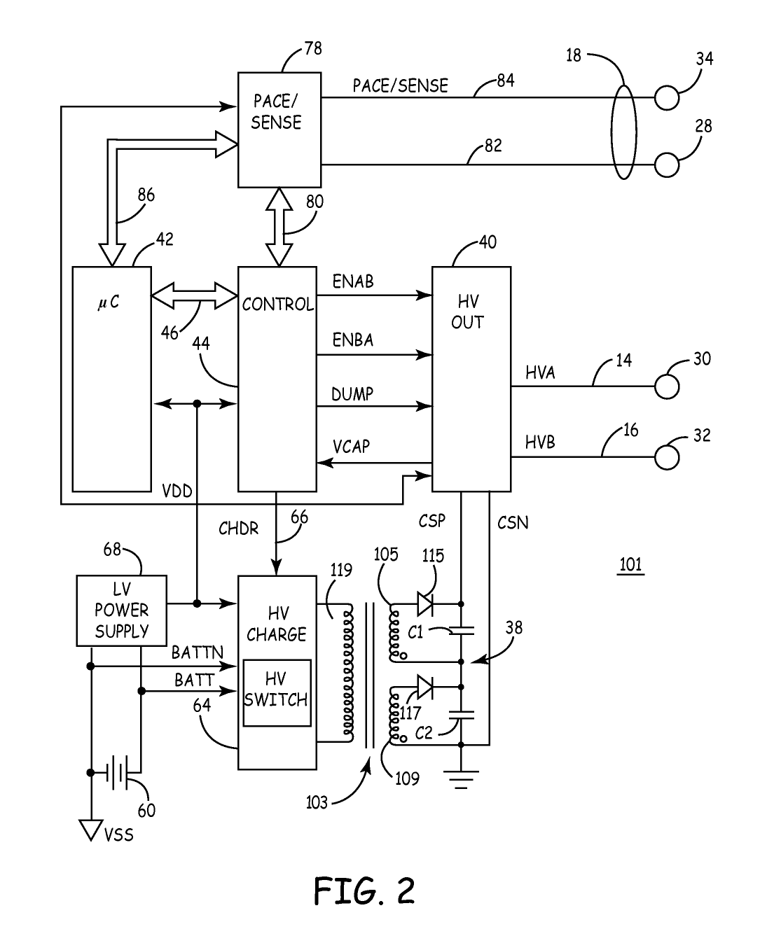

The ICD is typically formed having a housing that is hermetically sealed and is consequently impervious to body fluids. The ICD also includes a connector header for making electrical and mechanical connection with one or more leads bearing pacing, sensing and/or cardioversion/defibrillation electrodes adapted to be located in or around selected chambers of the heart. The housing is typically formed of a suitable body-compatible material approved for medical use, such as titanium. Typically, the housing is formed having major opposed or parallel surfaces joined together by sides enclosing an interior housing chamber or cavity and having electrical feed-throughs extending therethrough and into the connector header. The housing cavity receives the battery and the high voltage (HV) and low voltage (LV) electronic circuitry which can comprise ICs, hybrid circuits and discrete components, e.g. a step-up transformer and at least one high voltage output capacitor. Although, there is no particular preferred embodiment of such an ICD, FIGS. 1 and 2 depict one exemplary ICD in which the present invention can be advantageously implemented.

In FIG. 1, an ICD 10 and associated cardioversion/defibrillation leads 14, 16 and 18 are illustrated in relation to a patient's heart 12. The ICD 10 comprises a hermetically sealed, metallic housing 36 and a multi-lumen connector header 24 that contains separate connector blocks and ports for receiving, and for electrically and mechanically attaching, proximal connector ends of the leads 14, 16 and 18. Non-illustrated feed-throughs extend from connector blocks within the connector header 24, providing external electrical contact to high voltage and low voltage circuitry within the housing 36 in a manner that is conventional in the art.

The cardioversion/defibrillation leads 14, 16 and 18 bear relatively large surface area cardioversion/defibrillation electrodes 30, 32 and 26, respectively, that are located in, on, or about the heart 12. The cardioversion/defibrillation lead 14 extends subcutaneously and terminates distally in a subcutaneous electrode 30, which is intended to be mounted subcutaneously in the region of the left chest. The cardioversion/defibrillation lead 16 extends transvenously and terminates distally in an elongated coil electrode 32 that is located in the coronary sinus and great vein region of the heart 12. The electrode 32 extends around the heart from a point within the coronary sinus opening to a point in the vicinity of the left atrial appendage. The ventricular cardioversion/defibrillation lead 18 extends transvenously and is provided with electrode 26, which in this case is an elongated coil, and is located in the right ventricular chamber of the heart 12. Cardioversion/defibrillation therapeutic discharges can be applied between selected cardioversion/defibrillation electrodes.

In an exemplary embodiment, the ICD 10 further incorporates atrial and/or ventricular electrogram (EGM) sensing capabilities for detecting and providing therapy for atrial and/or ventricular arrhythmias. The ventricular lead 18 also includes a ventricular pace/sense electrode 34 that takes the form of a helical coil and is screwed into the myocardial tissue of the right ventricle. The lead 18 may also include an additional pace/sense electrode 28 for near field ventricular EGM sensing, or a surface electrode on the ICD 10 may be paired with the helical coil electrode 34 for far field ventricular EGM sensing. Additional near field and/or far field atrial EGM sensing and atrial pacing capabilities can be provided using atrial pace/sense electrode pairs on the atrial lead 16 and/or the IPG 10. The invention is also applicable to multiple lead and electrode systems adapted for the treatment of the patient's arrhythmias.

In the illustrated system, ventricular cardiac pacing pulses are delivered between the helical pace/sense electrode 34 and the ring electrode 28. The pace/sense electrodes 28 and 34 are also employed to sense EGM signals characteristic of ventricular contractions. As illustrated, it is anticipated that the right ventricular cardioversion/defibrillation electrode 26 will serve as the common electrode during sequential and simultaneous pulse multiple electrode defibrillation regimens. For example, during a simultaneous pulse defibrillation regimen, high voltage therapeutic discharges would simultaneously be delivered between the cardioversion/defibrillation electrodes 26 and 30, and between the cardioversion/defibrillation electrodes 26 and 32. During sequential pulse defibrillation, it is envisioned that high voltage therapeutic discharges would be delivered sequentially between the cardioversion/defibrillation electrodes 30 and 26, and between the coronary sinus cardioversion/defibrillation electrode 32 and the right ventricular cardioversion/defibrillation electrode 26. Single pulse, two electrode defibrillation pulse regimens may be also provided, typically between the right ventricular cardioversion/defibrillation electrode 26 and the coronary sinus cardioversion/defibrillation electrode 32. Alternatively, single pulses may be delivered between the cardioversion/defibrillation electrodes 28 and 30. The particular interconnection of the cardioversion/defibrillation on electrodes to the ICD will depend somewhat on the specific cardioversion/defibrillation pulse regimen that is employed.

FIG. 2 is a block diagram illustrating an ICD system 101 having a conventional high voltage, single core, step-up transformer 103. The ICD system 101 is merely exemplary of a variety of single chamber and dual chamber ICD systems having all or some of the capabilities described above, and in which the invention can be implemented. The exemplary ICD system 101 includes a battery 60, a DC-DC converter comprising a HV charging circuit 64, a HV single core transformer, a high-voltage (HV) output capacitor bank 38, and a HV output or discharge circuit 40 for discharging the charge on the HV output capacitor bank 38. The charge on the HV output capacitor bank 38, comprising series connected wet-tantalum capacitors C1 and C2 in this case, is selectively discharged through the cardioversion/defibrillation electrodes 26, 30 and 32 that are coupled to the HV out circuitry 40 by way of the leads 18, 14, 16, respectively. Similar ICD systems to that depicted in FIG. 2 in which the present invention can be implemented are shown, for example, in U.S. Pat. Nos. 4,830,006, 4,693,253, 4,971,058, 5,312,441, and 5,827,326.

The exemplary ICD system 101 illustrated in FIG. 2 is powered by the battery 60, coupled to the HV charging circuit 64 and to a power supply 68 that provides regulated power to the LV ICs, hybrid circuits, and discrete system components. An exemplary battery 60 is a lithium silver vanadium battery that is capable of providing the HV capacitor charging current. Such a battery 60 is capable of providing a voltage from about 3.2 volts when fresh to about 2.5 volts at a specified end of service.

The LV ICs and hybrid circuits powered by a supply voltage VDD (and other regulated voltages generated by LV power supply 68 in certain instances) comprise at least the illustrated microcomputer 42, the control circuitry 44, and the pace/sense circuitry 78. The hybrid circuits may also include other circuits, e.g. a system clock, power-on-reset circuitry, telemetry circuitry, physiologic and activity sensing circuitry. The LV supply voltage VDD is also applied to the HV charging circuit 64 to power the DC-DC conversion switching circuits and to the HV output circuitry 40 to power operation of certain circuitry therein.

The ICD system 101 is controlled by the operation of the microcomputer 42 and control circuitry 44 following an operating program, stored in ROM and RAM, which performs all necessary computational and control functions. The microcomputer 42 is linked to the control circuitry 44 by means of a bi-directional data/control bus 46 and further interrupt and signal lines (not shown), and thereby controls operation of the HV output circuitry 40 and the HV charging circuitry 64. Pace/sense circuitry 78 awakens the microcomputer 42 to perform any necessary mathematical calculations, to perform tachycardia and fibrillation detection procedures, and to update the time intervals controlled by the timers in the pace/sense circuitry 78 and the control circuitry 44. These functions may be performed upon receipt of a reprogramming command, or on the occurrence of signals indicative of delivery of cardiac pacing pulses or the sensing of selected features of the EGM characteristic of cardiac contractions. The basic operation of such a system in the context of an implantable pacemaker/cardioverter/defibrillator may correspond to any of the systems known to the art, and in more particular may correspond generally to those illustrated in the previously-referenced '006, '253, and '441 patents, for example.

The pace/sense circuitry 78 includes an R-wave sense amplifier such as that described in the previously-referenced '588 patent. The pace/sense circuitry 78 also includes a pulse generator for generating cardiac pacing pulses that may also correspond to any known cardiac pacemaker output circuitry. In addition, the pace/sense circuitry 78 includes timing circuitry for defining ventricular pacing intervals, refractory intervals and blanking intervals, under control of a microcomputer 42 by way of a control/data bus 86. Control signals that trigger generation of cardiac pacing pulses using the pace/sense circuitry 78, and signals indicative of the occurrence of R-waves from the pace/sense circuitry 78, are communicated to control circuitry 44 by means of a bidirectional data bus 80. The pace/sense circuitry 78 is also coupled to ventricular pace/sense electrodes 28 and 34, illustrated in FIG. 1, by means of conductors 82 and 84 in the ventricular lead 18; the coupling allows for bipolar sensing of R-waves and for delivery of bipolar pacing pulses to the ventricle of the heart 12. As previously noted, dual chamber or single chamber atrial pacing and sensing functions can also or alternatively be provided by employing suitable pace/sense circuitry 78 and suitable far field (unipolar) or near field (bipolar) atrial electrode pairs.

In the embodiment illustrated in FIG. 2, the HV output circuitry 40 is coupled to the output capacitor bank 38, including the capacitors C1 and C2, and is programmed to deliver biphasic cardioversion/defibrillation shocks to selected electrodes. The output capacitors C1 and C2 are coupled to the secondary windings 105 and 109 of the step-up transformer 103 by way of the diodes 115 and 117. The primary winding 119 of the step-up transformer 103 is coupled to the HV charging circuitry 64. The HV charging circuitry 64 is controlled by the CHDR signal on line 66 supplied by the control circuitry 44 when a malignant arrhythmia subject to cardioversion/defibrillation therapy is detected. The output capacitors C1 and C2 are charged by oscillations of the high frequency. The CSP and CSN voltage across the capacitor bank 38 is monitored and applied by way of the VCAP signal on line to the control circuitry 44. The control circuitry 44 detects the point when the VCAP signal level matches the programmed energy level of the cardioversion/defibrillation high voltage therapeutic discharge to be delivered. When that condition is satisfied, the control circuitry 44 terminates the CHDR signal and commences the operations to deliver the biphasic cardioversion/defibrillation shock to the selected cardioversion/defibrillation electrodes.

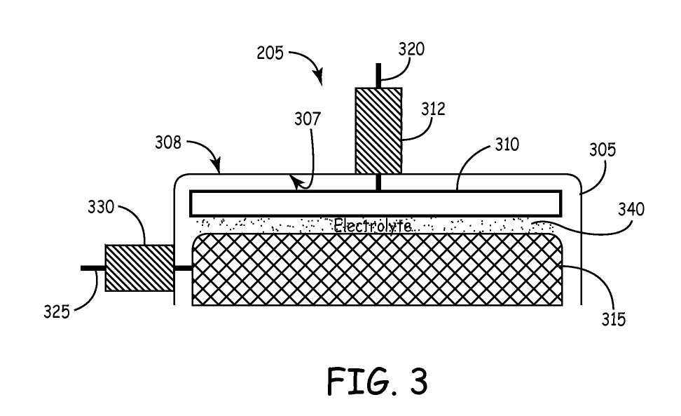

Turning now to FIG. 3, a more detailed, cross-sectional view of one of the capacitors C1, C2 disposed within the capacitor bank 38 is shown in accordance with one embodiment of the invention. For purposes of clarity and ease in illustrating the present invention, only a portion of the capacitor is shown in FIG. 3. In this particular embodiment, the capacitor, designated as 205 in FIG. 3, includes a hermetically sealed container 305 for encasing the capacitor internal contents. An exemplary container 305 is constructed from titanium, although the container 305 may be constructed from various other materials such as tantalum or niobium. The capacitor 205 further comprises a cathode 310, which takes the form of a metal conductive body that is disposed within the container 305. An exemplary cathode 310 is electrically isolated from an inner surface 307 of the container 305.

The cathode 310 is electrically coupled to a cathode lead 320 that extends through the container inner surface 307, and through a container outer surface 308. The cathode lead 320 is electrically isolated from the container 305 by a feed-through 312. In one embodiment, the feed-through 312 is constructed of a glass insulator that seals the cathode lead 320 to the container 305 while electrically isolating the cathode lead 320 from the container 305. The feed-through 312 also substantially prevents materials, such as fluid electrolytes, from leaking out of the container 305 while further substantially preventing foreign substances from entering into the container 305, thus reducing the likelihood of contamination of the container's 305 internal components.

The capacitor 205 is further configured with an anode 315 that is disposed within the container 305. In one embodiment, the anode 315 is constructed of tantalum. However, the anode 315 may alternatively be constructed of other valve metal materials, such as aluminum, niobium, zirconium, titanium, and alloys and oxides of these metals. Various other conductive materials may be used for the anode 315 without departing from the scope of the present invention.

The anode 315 is electrically coupled to an anode lead 325 that passes through the inner and outer surfaces 307, 308 of the container 305 by way of a feed-through 330. The feed-through 330, which may be similar in construction to the previously-discussed feed-through 312, electrically isolates the anode lead 325 from the container 305 in substantially the same manner that the feed-through 312 electrically isolates the cathode lead 320 from the container 305.

The container 305 is filled with a fluid electrolyte 340, which is disposed between and in contact with the anode 315 and the cathode 310. The electrolyte 340 provides a current path between the anode 315 and the cathode 310. The electrolyte 340 may include a water and glycol ether mixture, phosphoric acid, or other constituents of capacitor electrolytes known in the art. The selection of the particular electrolyte 340 may depend on the reactivity of the electrolyte 340 with the materials used for the anode 315 and the cathode 310. For example, a sulfuric acid solution used as the electrolyte may be desirable when the anode 315 is composed of tantalum.

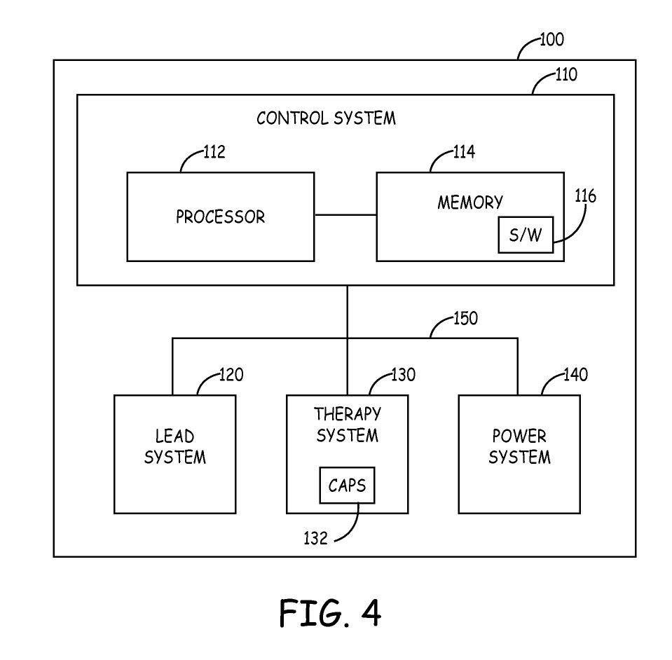

FIG. 4 is a block diagram that represents the basic components of the exemplary ICD 10. The ICD 10 includes several systems, components of which have been previously discussed. Generally speaking, the ICD systems include a control system 110, a lead system 120, a therapy system 130, a power system 140, and an inter-connective bus 150. The control system 110 includes a processor 112 and a memory 114. The memory 114 includes one or more software modules that store and execute one or more computer instructions for performing the ICD functions, one of the modules being a capacitor reformation programming module 116. Some embodiments of the invention replace the software modules, including the capacitor reformation programming module 116, with one or more hardware or firmware modules.

An exemplary lead system 120 includes one or more electrically conductive leads, such as atrial, ventricular, or defibrillation leads, that are suitable for insertion into a heart. As previously discussed, at least one of the leads is adapted for sensing electrical signals from a portion of the heart, and at least one of the leads is adapted for transmitting therapeutic doses of electrical energy. The lead system 120 also includes associated sensing and signal-conditioning electronics, such as atrial or ventricular sense amplifiers and/or analog-to-digital converters.

Other exemplary lead systems 120 support ventricular epicardial rate sensing, atrial endocardial bipolar pacing and sensing, ventricular endocardial bipolar pacing and sensing, and epicardial patches. Some exemplary lead systems 120 also support two or more pacing regimens. Also, in some embodiments such as monopolar stimulation assemblies, at least a portion of the ICD housing 36 is used as an electrode. The invention, however, is not limited in terms of lead or electrode types, lead or electrode configurations, pacing modes, sensing electronics, or signal-conditioning electronics.

The therapy system 130 includes a capacitor system 132 and other previously-discussed circuitry for delivering or transmitting electrical energy in measured doses through the lead system 120 to a heart or other body tissue. Additionally, the therapy system 130 includes one or more timers, analog-to-digital converters, and other non-illustrated conventional circuitry for measuring various electrical properties related to performance, use, and maintenance of the therapy system.

In general operation, the lead system 120 senses atrial or ventricular electrical activity and provides data representative of this activity to the control system 110. Within the control system 110, the processor 112 processes the data according to instructions from the software modules. If appropriate, the processor 112 then directs or causes the therapy system 130 to deliver one or more measured doses of electrical energy or other therapeutic agents through the lead system 120 to a targeted body tissue. Additionally, the capacitor reformation programming module 116 includes one or more instructions for maintaining the capacitors 132, as will be discussed in further detail.

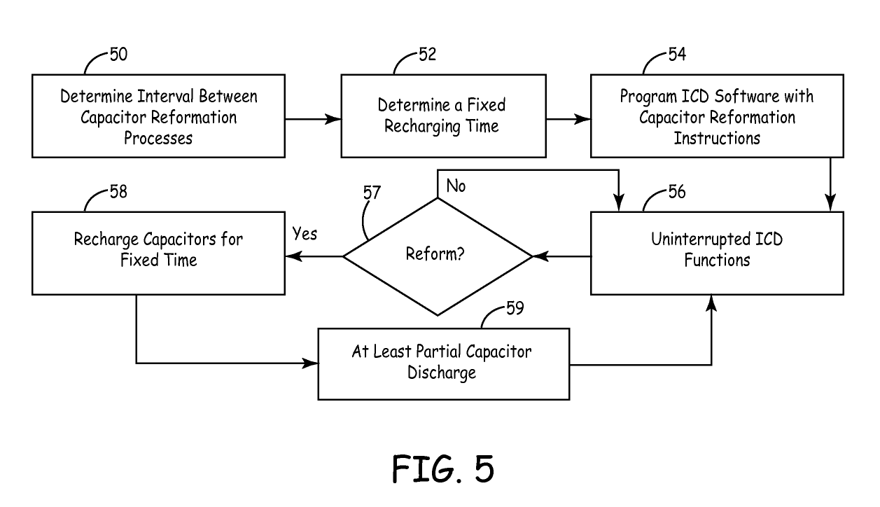

Referring now to FIG. 5, a method of scheduling and executing capacitor reformation maintenance for an ICD is outlined in a block diagram. Although the method is described in the context of an ICD, the same capacitor reformation scheduling and execution steps may be applied to other devices that utilize high-voltage capacitors to store a charge for stimulating body tissue. Further, the present method may be used for reformation of a wide variety of different capacitors, including electrolytic capacitors. Exemplary electrolytic capacitors that may be reformed in accordance with the present method include tantalum capacitors. Wet tantalum capacitors are one type of tantalum capacitor that particularly benefit from the capacitor reformation process of the present invention.

The method begins with block 50, which entails determining the appropriate intervals at which capacitor reformation should be executed. Although capacitor reformation provides assurance that the capacitors 132 are fully charged and functioning properly, the reformation process also provides an opportunity to evaluate the charging system, including the capacitors 132 and the power system 140 for efficiency and adequacy.

Factors that are significant when determining how often the capacitors 132 should be reformed include the characteristics of the capacitor and its components, and also the characteristics of the battery that is being used to recharge the capacitors 132. The interval between capacitor reformations is a programmable feature, and in an exemplary method, the interval is a fixed time period, for example a one-month period, a three-month period, a six-month period and so forth, that is programmed into the capacitor reformation programming module 116. For capacitors that have highly stable dielectric material, the interval between capacitor reformations may be fixed at six months or longer. For capacitors that theoretically, or based on empirical data, have a less stable dielectric material, the interval between capacitor reformations may be fixed at one month, for example.

An indicator that influences a time interval between capacitor reformations is the amount of time it takes to charge a capacitor. In an exemplary embodiment, if the charge time is faster than a predetermined threshold value, i.e. sixteen seconds, then the interval between capacitor reformations is fixed at six months, for example. If the charge time is slower than the predetermined threshold value, then the interval between capacitor reformations is fixed at one month, for example.

Continuing with the method outlined in FIG. 2, block 52 entails determining a fixed capacitor recharging time period. Prior to the present invention, electrolytic capacitors such as tantalum would be reformed as necessary by charging the capacitors at a relatively high voltage. Conventionally, capacitors have been charged to voltages in the range of 90% to 100% of the maximum operating voltage of the capacitor. One advantage provided by the present method lies in the discovery that charging the capacitors for a fixed period of time at a relatively low voltage is just as effective as charging at or near the capacitor's maximum voltage. Using the present method, a fixed capacitor reformation charging time period is determined and programmed into the capacitor reformation programming module 116 based on a database of empirical data, including charging data, for the particular capacitors and power source that an ICD employs. The empirical data may include chemical and physical characteristics for the capacitors and the power source, and data from laboratory tests during which similar or identical capacitors were charged for various time periods to determine optimal recharging times and voltages. In an exemplary embodiment, a capacitor is charged to a voltage that is at least 20% and less than 90% of the capacitors' maximum rated voltage, and preferably to about 50% of the capacitor's maximum rated voltage. The fixed time at which charging is performed during capacitor reformation may also be determined to be a percentage of the minimum capacitor first charge time, which is determined at the capacitor's beginning of life. In another embodiment, the fixed time can be reset after a therapeutic treatment and subsequent recharge, and set as a percentage of the time of the last recharge following a therapeutic treatment.

Establishing a fixed charging period enables the use of simplified circuitry and software configurations because software and circuitry directed to monitoring the voltage during reformation, and to preventing capacitor damage due to overcharging, can be removed. Further, if the capacitor or the power source degrade over time and are consequently unable to reach a once-obtainable voltage, then programming and circuitry would conventionally be required to instruct the power system to stop charging in order to save energy and avoid power source depletion. Under the principles of the present invention, such circuitry is also unnecessary since charging is performed for a fixed period of time without attempting to approach the capacitor's maximum voltage.

Block 54 entails programming the capacitor reformation programming module 116 in the ICD 100 with capacitor reformation instructions, including the predetermined fixed intervals and time period for charging the capacitors during the capacitor reformation process. To minimize software and circuitry complexity, the capacitor reformation programming module 116 is configured to automatically perform a capacitor reformation procedure every time the fixed time period lapses. In another exemplary embodiment, if the fixed time period is interrupted by an event that causes the capacitors to discharge and recharge, e.g. a therapeutic burst of current, then the capacitor reformation programming module 116 simply restarts a timer that elapses when the fixed time period lapses. Further, to minimize circuitry and software complexity, the capacitor reformation programming module 116 is configured to perform the capacitor reformation process for the previously determined fixed time period, based on the previously-discussed empirical data and other factors such as the first charge time at the capacitor beginning of life, and the charge time following the most recent therapeutic treatment.

The determining and programming steps 50, 52, 54 are initially performed during the ICD assembly. However, after implantation, the same steps may be performed as a physician or other user perceives the need to modify the fixed interval between capacitor reformation processes, or the time and/or voltage at which the capacitor reformation process is performed.

At block 56, the ICD performs its functions in an uninterrupted manner. The ICD functions may include monitoring the patient's heart for irregular cardiac rhythm, and providing stimulation to the heart as necessary. The ICD functions also include determining whether capacitor reformation is necessary.

If, in decision block 57, the processor 112 determines that the fixed time interval between capacitor reformation processes has elapsed, then the method proceeds to block 58, which entails recharging the capacitor for the fixed time period. If reformation of the capacitors is not necessary, the method returns to block 56 as the ICD continues its uninterrupted functions.

After performing the capacitor reformation process in block 58, the method proceeds to block 59, which entails at least partially discharging the capacitors. The discharge may be performed through any of a variety of techniques, including discharging through non-therapeutic channels such as through a resistance, including a resistor or other resistive load, impedance, and so forth. Further, the capacitors may be discharged through an open circuit bleed, that is, through natural leakage current in the system. Typically, if the discharge is through a non-therapeutic load, the discharge may occur relatively rapidly. However, if the discharge is through leakage current, the leakage current will occur over a prolonged period depending on the characteristics of the system. After at least partially discharging the capacitors, the method returns to block 56 for performance of uninterrupted ICD functions.

The previously-discussed embodiments of an implantable medical device, and a method of reforming capacitors in such a device, overcome problems associated with degraded charging efficiency for various types of capacitors. In addition, the implantable medical device and the capacitor reforming methods have improved efficiency in terms of power consumption, and have improved simplicity in their design as they require minimal programming and circuitry.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

1. A method of reforming one or more capacitors in an implantable medical device having a control system that includes a programming module, each capacitor in the implantable medical device having a maximum-charging voltage, the method comprising the steps of:
using empirical capacitor charging data, determining a capacitor reformation time period;
configuring the programming module with capacitor reformation instructions that include the capacitor reformation time period; and
in response to the capacitor reformation instructions, charging the capacitors, for the capacitor reformation time period, to a voltage that is at least 20% and less than 90% of the maximum-charging voltage.

2. The method according to claim 1, further comprising the step of:
allowing the capacitor to at least partially discharge through leakage current.

3. The method of claim 1, wherein the step of charging the capacitors includes charging to between 25% and 75% of the maximum-charging voltage for the capacitors.

4. The method of claim 1, wherein the step of charging the capacitors includes charging to about 50% of the maximum-charging voltage for the capacitors.

5. The method of claim 1, wherein the capacitor reformation time is calculated as a percentage of a beginning-of-life charge time for the capacitors.

6. The method of claim 1, wherein the capacitor reformation time is calculated as a percentage of the most recent charge time for the capacitors.

7. The method of claim 1, wherein the empirical charging data comprises laboratory tests on the capacitors to determine optimal capacitor reformation time period.

8. The method of claim 1, wherein the capacitor reformation instructions further include a time interval to begin after charging the capacitors, and to elapse when the capacitors are to be recharged.

9. An implantable medical device, comprising:
a control system having a processor and a programming module configured with capacitor reformation instructions that include a fixed capacitor reformation time period during which the capacitors are to be charged to a reformation voltage that is at least 20% and less than 90% of the maximum-charging voltage;
a lead system comprising at least one lead; and
a therapy system, comprising a plurality of capacitors having a maximum-charging voltage, and coupled to receive the capacitor reformation instructions and, responsive thereto, to charge the plurality of capacitors for the fixed capacitor reformation time period, to the reformation voltage.

10. The implantable medical device of claim 9, wherein the reformation voltage is between 25% and 75% of the maximum-charging voltage for the capacitors.

11. The implantable medical device of claim 9, wherein the reformation voltage about 50% of the maximum-charging voltage for the capacitors.

12. The implantable medical device of claim 9, wherein the capacitor reformation time is a percentage of a beginning-of-life charge time for the capacitors.

13. The implantable medical device of claim 9, wherein the capacitor reformation time is a percentage of the most recent charge time for the capacitors.

14. The implantable medical device of claim 9, wherein the fixed capacitor reformation time period, are predetermined values based on empirical charging data that comprises laboratory tests on the capacitors to determine optimal capacitor reformation voltages and times.

15. The implantable medical device of claim 9, wherein the capacitor reformation instructions further include a time interval to begin after charging the capacitors, and to elapse when the capacitors are to be recharged.

16. A method of reforming one or more capacitors in an implantable medical device having a control system that includes a programming module, each capacitor in the implantable medical device having a maximum-charging voltage, the method comprising the steps of:
using empirical capacitor charging data, determining a capacitor reformation time period;
configuring the programming module with capacitor reformation instructions that include the capacitor reformation time period, and a time interval to begin after charging the capacitors, and to elapse when the capacitors are to be recharged;
when the time interval elapses, and in response to the capacitor reformation instructions, charging the capacitors, for the capacitor reformation time period, to a voltage that is at least 20% and less than 90% of the maximum-charging voltage; and
allowing the capacitor to at least partially discharge through leakage current.

17. The method of claim 16, wherein the step of charging the capacitors includes charging to between 25% and 75% of the maximum-charging voltage for the capacitors.

18. The method of claim 16, wherein the step of charging the capacitors includes charging to about 50% of the maximum-charging voltage for the capacitors.

19. The method of claim 16, wherein the capacitor reformation time is calculated as a percentage of a beginning-of-life charge time for the capacitors.

20. The method of claim 16, wherein the capacitor reformation time is calculated as a percentage of the most recent charge time for the capacitors.
